(19) United States
(12) Patent Application Publication
(10) Pub. No.: US 2014/0325746 A1
(43) Pub. Date: Nov. 6, 2014

(54) FLUID VOIDING APPARATUS WITH VACUUM DEVICE

(71) Applicant: FemMed, Inc., Minnetonka, MN (US)

(72) Inventor: James C. Block, Maple Plain, MN (US)

(21) Appl. No.: 14/361,632

(22) PCT Filed: Nov. 30, 2012
(86) PCT No.: PCT/US12/67248

§ 371 Date: May 29, 2014

Related U.S. Application Data
(60) Provisional application No. 61/565,807, filed on Dec. 1, 2011.

(51) Int. Cl. A61F 5/44; A61F 5/455; A61F 5/453

(52) U.S. Cl. CPC A61F 5/44; A61F 5/453; A61F 5/455
USPC 4/144.3

(57) ABSTRACT

An external fluid voiding apparatus that may be configured for either female or male use includes a container, a conduit and a vacuum device, which define a urine drainage passage. The vacuum device may be deformed externally so as to create a vacuum effect inside the apparatus when the apparatus is fitted around the female's vulval region or the male's penis. The vacuum effect facilitates drawing urine from a reservoir of the container to the vacuum device through the conduit. The urine may be collected and stored in the vacuum device. A user may drain the urine collected inside the vacuum device through a drainage port. Alternatively, a user may disconnect a used vacuum device and replace it with a clean one.

### FIELD OF TECHNOLOGY

The device disclosed here relates generally to fluid voiding apparatuses. More particularly, the device relates to external fluid voiding apparatuses with a vacuum device for male or female external use.

### BACKGROUND

A variety of sanitary devices have been designed to allow males or females to void urine while standing up in order to avoid non-hygienic contact, for example, with toilet facilities. Such devices often include a funnel shaped collector, a fluid passage and a collection bag. Some devices are configured to be fitted around a female's urethra, such as a urine catheter disclosed in U.S. Pat. No. 6,280,425. To use these devices, a user has to insert at least a portion of the devices into her vulval and fits an opening of the catheter around her urethra. These devices are more difficult to use and may cause problems such as infections. Some other devices are configured to be fitted externally around a female's vulval region. However, generally a user has to hold such a device when in use. To facilitate draining urine collected in the collector, a vacuum source sometimes can be used with the fluid voiding device to create a vacuum effect in the fluid passage to facilitate the fluid flow.

U.S. Pat. No. 5,911,222 discloses an external liquid removal system that includes a urine collector, tubing, a vacuum source and a collection bag. The system can turn on a vacuum source when the collector collects liquid and drains the liquid in the collector to the collection bag. In this system, the vacuum source may be in communication with the fluid drainage passage to create a vacuum effect in the drainage passage to facilitate fluid drainage through the passage, but the vacuum source is connected to the fluid drainage passage via a side branch and is not positioned in-line as a part of the fluid drainage passage.

Urinary voiding devices have also been configured for male use. U.S. Pat. No. 4,957,487 discloses an external male urinary voiding device that includes a penile sheath, tubing and an evacuator device. The sheath can be fitted to the external of a male penis, and the evacuator device may be used to create a vacuum effect to enlarge the penis and facilitate urine flow through the tubing. The urine may be collected by the evacuator device. However, the evacuator device disclosed may contain a complicated internal mechanism, such as springs disposed inside an interior chamber of the evacuator device, to create the vacuum effect, and therefore may be difficult to be cleaned after usage. In use, the user generally wears the evacuator device around the user's thigh. Because the tubing connects the penile sheath and the evacuator device, the length of the tubing may limit the mobility of the user. Furthermore, because the evacuator device is generally worn inside a user's pants, the user may have difficulty in accessing the device in use.

Although these devices may be suitable for their intended uses, they may have complicated structures and often can be difficult to use and clean after use. Improvements may be made in fluid voiding apparatuses.

### SUMMARY

An external fluid voiding apparatus as disclosed herein has a vacuum device positioned in-line as a part of a urine drainage passage. The external voiding apparatus may have a container that is configured to form a generally air-tight seal around a perineal area of a human being, such as a female vulval region or a male penis, and a vacuum device positioned in line with a urine drainage passage. The vacuum device may be configured to create and maintain a vacuum effect in the urine drainage passage to facilitate drawing urine toward the vacuum device.

In some embodiments, the vacuum device may be employed as a collecting device that can collect and store urine. The vacuum device can be easily cleaned or exchanged after usage.

In some embodiments, the fluid voiding apparatus may include a container that has a top portion and a reservoir. The top portion is configured to fit a perineal area of a human being, such as a vulval region of a female or a penis of a male. The reservoir may be configured to receive fluid, such as urine, and is in fluid communication with the drainage passage.

In some embodiments, at least a portion of the vacuum device may be elastic and can be deformed externally. The vacuum device may be configured to have a non-activated state and an activated state. The vacuum device may be configured to have an internal volume. The deformation of the elastic portion by an external force activates the vacuum device and reduces the internal volume. The vacuum device in the activated state may create a vacuum effect in the drainage passage so that fluid in the drainage passage may be drawn toward the vacuum device. When the deformed elastic portion returns to its configuration before the deformation, the vacuum device is in a non-activated state, in which the vacuum effect in the drainage passage may be released.

In some embodiments, at least a portion of the side wall of the vacuum device may include bellow folds that are elastic. The vacuum device may be activated by compressing the bellow folds with an external force, and the elasticity of the bellow folds may enable the bellow folds to return to the non-activated state as fluid is received by the vacuum device to release the vacuum created.

In some embodiments, the vacuum device may include an one way valve, such as a duck bill valve, that may prevent the urine collected inside the vacuum device from flowing back to the conduit. The vacuum device may also include a drainage port and a cap covering a drainage port, which may be removed to release air or urine collected inside the vacuum device.

In some embodiments, the whole body of the vacuum device may be made of an elastic material. When an external force is applied to deform the elastic material of the vacuum device, the elasticity of the elastic material can create a tendency of returning to the non-activated state without the external force so as to create a vacuum effect inside the urine drainage passage.

In some embodiments, the top portion may be equipped with an air bladder that is configured to generally follow the anatomy of a female's vulval region. The air bladder may include one or more internal air chambers. The air chamber may include a valve from which a user may add or release the air in the chamber. By increasing or reducing the air inside the air chamber, a user may change a hardness of the air chamber.

In some embodiments, the air chamber is configured to be fitted to a female vulval region directly. In some embodiments, an adhesive may be applied to at least a portion of the air bladder to facilitate creating a generally air-tight seal between the air bladder and the female vulval region.

In some embodiments, the air bladder may be disposed directly underneath a surface of the container that extends inwardly. The inwardly extended surface may prevent urine from splashing out of the open side of the container. In some embodiments, the hardness of the air bladder may be regulated by increasing or reducing air inside the air chamber of the air bladder, and therefore the firmness of the surface may be regulated.

In some embodiments, the container may have a small profile and a small reservoir. In one embodiment, the conduit may be oriented generally parallel to the open side of the container. In one embodiment, the vacuum device may be positioned on the same side of the open side of the container relative to a drainage opening between the reservoir and the conduit.

In some embodiments, the structure of the external voiding apparatus may include structures that are configured to enhance the rigidity of the external voiding apparatus, which may help the external voiding apparatus maintain its shape when the vacuum effect is created. In one embodiment, the conduit may include rib-like structure and an external profile that enhance the rigidity of the conduit.

In some embodiments, the container may be configured for male use. One embodiment of a container configured for male use may have a sheath that is configured to surround a male's penis. In one embodiment, the sheath may include flaps that are configured to form an air-tight closure. A male user may put the sheath around his penis and close the flaps to form a generally air-tight seal between the sheath and the penis.

### BRIEF DESCRIPTION OF THE DRAWINGS

Referring now to the drawings in which like reference numbers represent corresponding parts throughout.

FIG. 1 illustrates a perspective view of one embodiment of an external fluid voiding apparatus that is configured for female use.

FIG. 2 illustrates a side section view of one embodiment of a vacuum device.

FIG. 3 illustrates an elevated top plan view of a container and a portion of a conduit of one embodiment of an external fluid voiding apparatus.

FIG. 4 illustrates an elevated top perspective view of a container and a portion of a conduit of another embodiment of an external fluid voiding apparatus configured for female use.

FIGS. 5A and 5B illustrate a side perspective view and an elevated side perspective view of the external female fluid voiding apparatus of FIG. 4.

FIG. 6 illustrates a top perspective view of a container and a portion of a conduit of another embodiment of an external fluid voiding apparatus configured for female use.

FIG. 7 illustrates a perspective view of another embodiment of an external fluid voiding apparatus.

FIG. 7A illustrates an enlarged section view taken along line 7A-7A in FIG. 7.

FIG. 8 illustrates a perspective view of one embodiment of an external fluid voiding apparatus configured for male use.

### DETAILED DESCRIPTION

In the following description of the illustrated embodiments, an external voiding fluid voiding apparatus with a vacuum device is described. The apparatus may be configured for male or female external use. Reference is made to the accompanying drawings that form a part hereof, and in which is shown by way of illustration of the embodiments in which the apparatus may be practiced.

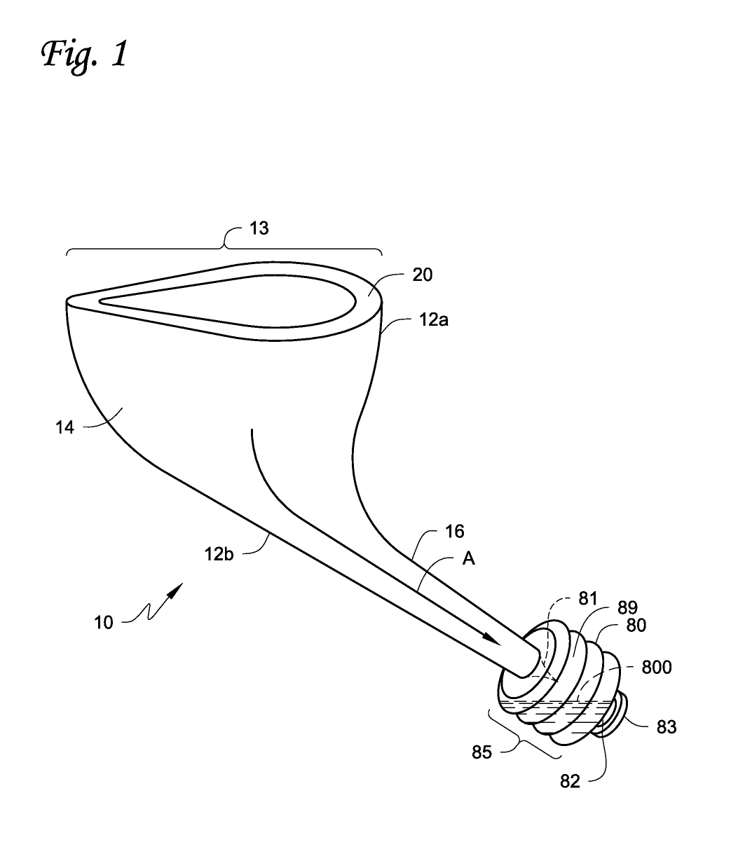

FIG. 1 shows one embodiment of a urine voiding apparatus 10 that may be configured for female use. The apparatus 10 includes a container 14 that has an open side 13, a conduit 16 and a vacuum device 80. The container 14 further includes a top portion 12a and a bottom portion 12b. A surface 20 may be connected to the top portion 12a of the container 14 and extends generally about a perimeter of the open side 13 defined by the container 14. The top portion 12a may be generally sized and shaped to cover a female vulva region externally. The bottom portion 12b is in fluid communication with the conduit 16. The conduit 16 is in fluid communication with the vacuum device 80. The arrow A is referred to as a fluid drainage passage that is defined by the conduit 16, and the vacuum device 80 respectively, and the direction toward which urine is drawn.

The vacuum device 80 is positioned in-line as a part of the drainage passage A. The phrase “in-line” generally means that the vacuum device 80 is part of the fluid drainage passage A, and is in fluid communication with the conduit 16. The vacuum device 80 may be configured so that urine drained through the fluid drainage passage A can be received by the vacuum device 80, and collected and stored in an internal chamber 89 of the vacuum device 80. The vacuum device 80 may also include a drainage port 82 and a cap 83 to close the drainage port 82. Urine 800 collected in the internal chamber 89 of the vacuum device 80 may be drained through the drainage port 82.

The vacuum device 80 may generally have a portion 85 that is elastic. The vacuum device 80 may have a non-activated state and an activated state. The elastic portion 85 may be deformed (or pressed) by an external force and the deformation of the elastic portion 85 can activate the vacuum device 80 to the activated state. The non-activated state is when the elastic portion 85 is generally not deformed by an external force. The internal chamber 89 has an internal volume. In the activated state, the internal volume decreases in comparison to the non-activated state; and the elastic portion 85 has a tendency to return to the configuration of the non-activated state; which creates a vacuum effect with a negative air pressure in the fluid drainage passage A compared to the normal atmosphere pressure. As urine is drained toward the internal chamber 89 by the vacuum effect, the internal volume increases and the vacuum device 80 returns to the non-activated state, releasing the vacuum effect inside the fluid drainage passage A.

The vacuum device 80 may have an one-way valve 81, which is configured to allow fluid, such as urine, to flow from the fluid drainage passage A to the vacuum device 80, but generally prevent the urine 800 collected in the vacuum device 80 to flow back to the fluid drainage passage A.

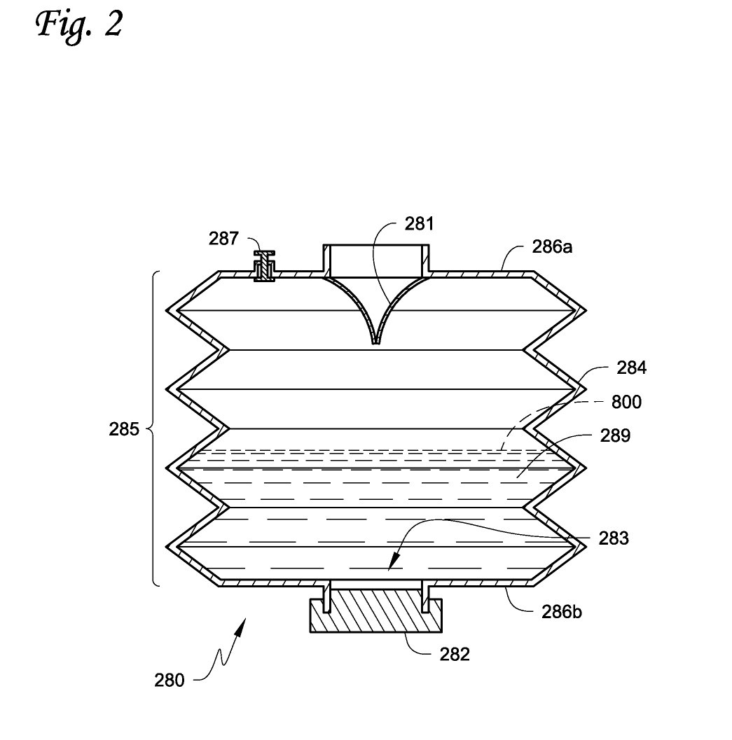

Now referring to FIG. 2, one embodiment of the vacuum device 280 will be described in greater detail. The vacuum device 280 may have a side wall 285. At least a portion of the side wall 285 has one or a plurality of bellow folds 284. The bellow folds 284 may be made of elastic materials, such as rubber. The vacuum device 280 has an internal chamber 289 that is configured to collect and/or store urine 800.

When uncompressed, the vacuum device 280 is in a non-activated state and the internal chamber 289 has an initial volume. The vacuum device 280 may be activated by deforming the bellow folds 284 by compressing the ends 286a and 286b toward each other with an external force, which decreases the distance between the neighboring bellow folds 284 and the volume of the internal chamber 289 of the vacuum device 280. A reduced volume of the internal chamber 289 may be reached when all the walls of the neighboring bellow folds 284 are pressed against each other. The bellow folds 284 may be configured to maintain their elasticity during the change from the initial volume to the reduced volume. When the external force is withdrawn, because the bellow folds 284 are elastic for example, they return to their non-activated state and the volume of the internal chamber returns to the initial volume. The vacuum device 280 may have a drainage port 283. The drainage port 283 may be closed by a drainage port cap 282. In one embodiment, the closure formed by the cap 282 and the drainage port 283 may be air-tight. The phrase “air-tight” generally means air impermeable, or air is generally incapable of penetrating. The vacuum device 280 may also have a one-way valve, such as an one-way valve 281 (such as a duck bill valve), that is configured to prevent urine 800 in the internal chamber 289 from flowing back out from the one-way valve 281. The vacuum device may also have an air valve 287 that is separate from the drainage port 283.

The vacuum device 280 may be made entirely with one elastic material, or the ends 286a and 286b or other sections of the vacuum device 280 may be made of materials that are different from the material of the bellow folds 284.

The embodiment illustrated in FIG. 2 is exemplary. It will be appreciated that the vacuum device may have other configurations. The vacuum device does not have to have bellow folds. A configuration of the vacuum device that may be deformed by an external force and return to its original configuration after withdrawal of the external force may satisfy. For example, the vacuum device may be made entirely with an elastic material such as rubber, or a portion of the vacuum device may be made with an elastic material. When an external force is applied to the elastic material, the elastic material is deformed but is capable of maintaining its elasticity during the entire deformation process. When the external force is withdrawn, the elastic material of the vacuum device may return to its original configuration.

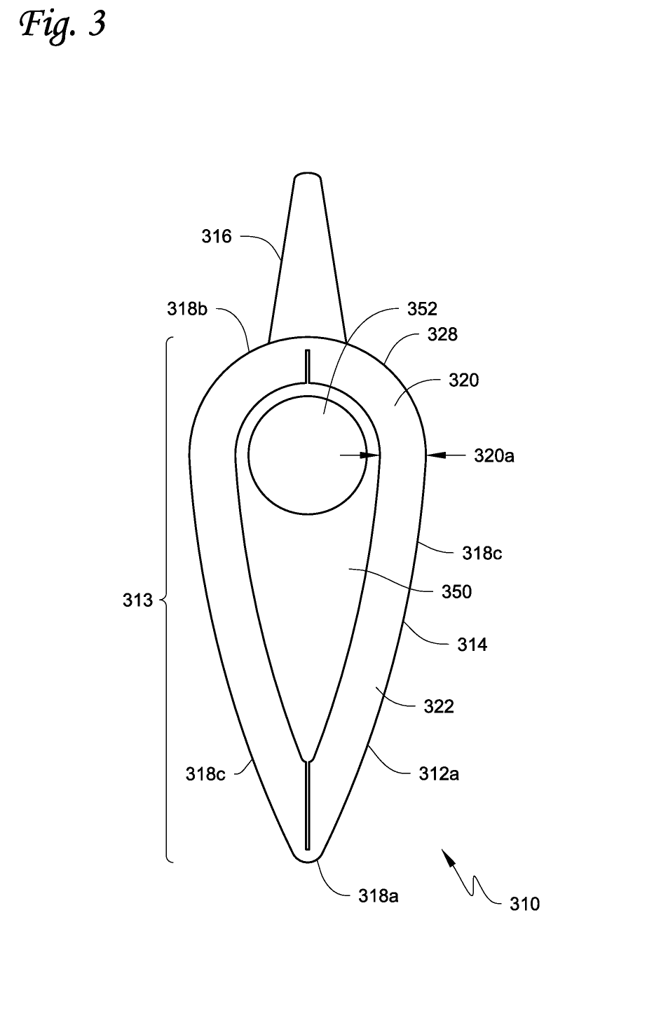

Referring to FIG. 3, an embodiment of an external voiding apparatus 310 with a container 314 configured for female use is described in greater detail. The female container 314 has a generally oval platform that defines the open side 313 and a reservoir 350 configured for receiving urine. A drainage opening 352 at the bottom of the reservoir 350 is in fluid communication with a conduit 316. The female container 314 includes a superior end 318b configured to align generally with the mons veneris of the female anatomy and an inferior end 318a configured to align generally with the perineal region of the female anatomy. Extending between the superior end 318b and the inferior end 318a of the container 314 are two outwardly curved sides 318c that are arranged and configured to generally align along the labium majus of the female anatomy. In one embodiment, an extra labia sealing structure such as a pressure sensitive adhesive 322 is disposed on a surface 320 of a top portion 312a. The extra labia sealing structure extends generally around the perimeter of the open side 313. The surface 320 includes a width 320a, where the width 320a provides a suitable contact area for the pressure sensitive adhesive 322 to be affixed to the surface 320 and thereby suitably contact a user's vulval region when the urine voiding apparatus 310 is in use. A raised ridge 328, substantially resembling a rib-like structure or protrusion, may be disposed proximate the top portion 312a of the container 314 on the surface 320 and about the perimeter of the outer surface of the container 314. The raised ridge 328 is adjacent to the pressure sensitive adhesive 322 and assists in preventing leakage and splashing from the urine voiding apparatus 310 when the apparatus is supported against the periphery of a user's vulval region during use. It will be appreciated that a raised ridge resembling raised ridge 328 may also be included on the surface 320 at an inner perimeter of the surface 320.

The conduit 316 can be connected to and configured to be in fluid communication with a vacuum device, such as the vacuum device 280 as illustrated in FIG. 2. Referring to FIGS. 2 and 3, in practice, a user may simply fit and press the open side 313 of the container 314 to the periphery of the vulvar region, which generally includes the tissues of the mons veneris, labium majus and the tissue of the perineum. The pressure sensitive adhesive 322 on the surface 320 may be configured to form a generally air-tight seal between the container 314 and a periphery of a user's external vulval region. Without deformation of the elastic bellow folds 284 of the vacuum device 280 by an external force, the vacuum device 280 is in a non-activated state. After the external voiding apparatus 310 is placed around the periphery of the vulval region, the user may activate the vacuum device 280 by removing the cap 282 of the drainage port 283, compressing the ends 286a and 286b of the vacuum device 380 toward each other to deform the bellow folds 284 and decrease the volume of the internal chamber 289 of the vacuum device 280, and then replacing the cap 282 to close the drainage port 283. Because the elasticity of the bellow folds 284 of the vacuum device 280 creates a tendency to return to their non-activated state without the external force, the vacuum device 280 creates a vacuum effect in the conduit 316 of the external voiding apparatus 310. Further, a user may activate the vacuum device 280 by compressing the ends 286a and 286b and releasing air from the air valve 287 without the need to open the cap 282 of the drainage port 283. The phrase “vacuum effect” generally means a state that the air pressure inside the drainage passage A is lower than the normal atmosphere pressure or the external air pressure of the vacuum device 280.

When the user releases urine, the urine is drawn toward the vacuum device 280 through a drainage passage, such as the fluid drainage passage A as shown in FIG. 2, due to the vacuum effect; and can be stored in the internal chamber 289 of the vacuum device 280. The vacuum effect in the conduit 316 of the external voiding apparatus 310 may facilitate drawing urine toward and into the vacuum device 280. The vacuum device 280 may also have the one way valve 281 configured to help prevent urine 800 collected in the vacuum device 280 from flowing back to the conduit 316. As urine is drawn into the internal chamber 289 of the vacuum device 280, the volume of the internal chamber 289 increases and the vacuum device 280 gradually returns toward the non-activated state. The vacuum effect in the drainage passage of the external voiding apparatus 310 may be sustained until the vacuum device 280 returns to the non-activated state. At that point, the air pressure inside the drainage passage is generally at equilibrium with the atmosphere or external environment. The user may drain the urine 800 collected inside the vacuum device 280 by removing the cap 282 on the drainage port 283. Or the user may disconnect the vacuum device 280 entirely from the external voiding apparatus 310 and replace it with another clean vacuum device. Because the one way valve 281 can prevent urine 800 from flowing backwards and the cap 282 closes the drainage port 283, the urine 288 collected in the removed vacuum device 280 does not leak out from the vacuum device 280 after removal. Therefore, the removed vacuum device 280 may be stored easily without the worry of urine leakage, and can be disposed or cleaned easily later. To facilitate the removal of the vacuum device 280, a quick release type connector (not shown) may be used to connect the vacuum device 280 to the conduit 316. The vacuum device 280 may have an air valve 287 that is separate from the drainage port 283. Besides being able to activate the vacuum device 280 by compressing air out of the vacuum device 280 through the air valve 287 as described above, a user may also terminate the vacuum effect in the vacuum device 280 by opening the air valve 287 to allow air to get into the vacuum device 280, so that the air pressure inside the vacuum device 280 is generally at equilibrium with the atmosphere or the external environment.

Because an external force may be used to activate the vacuum device 280 and therefore create the vacuum effect in the conduit 316 of the external voiding apparatus 310, the vacuum device 280 may be activated simply by compressing the elastic portion of the vacuum device 280 by hands. Furthermore, because a portion of the sidewall of the vacuum device 280 may be configured to create the vacuum effect, the internal structure of the vacuum device 280 may be simplified so that the vacuum device 280 may be cleaned easily after use. For this purpose, the internal chamber 289 may be simply an empty space inside. It will also be appreciated that because the vacuum device 280 may be removed from the external voiding apparatus 310, different sizes and shapes of the vacuum device 280 may be made so that a user may choose different configurations of the vacuum device 280 to fit her needs.

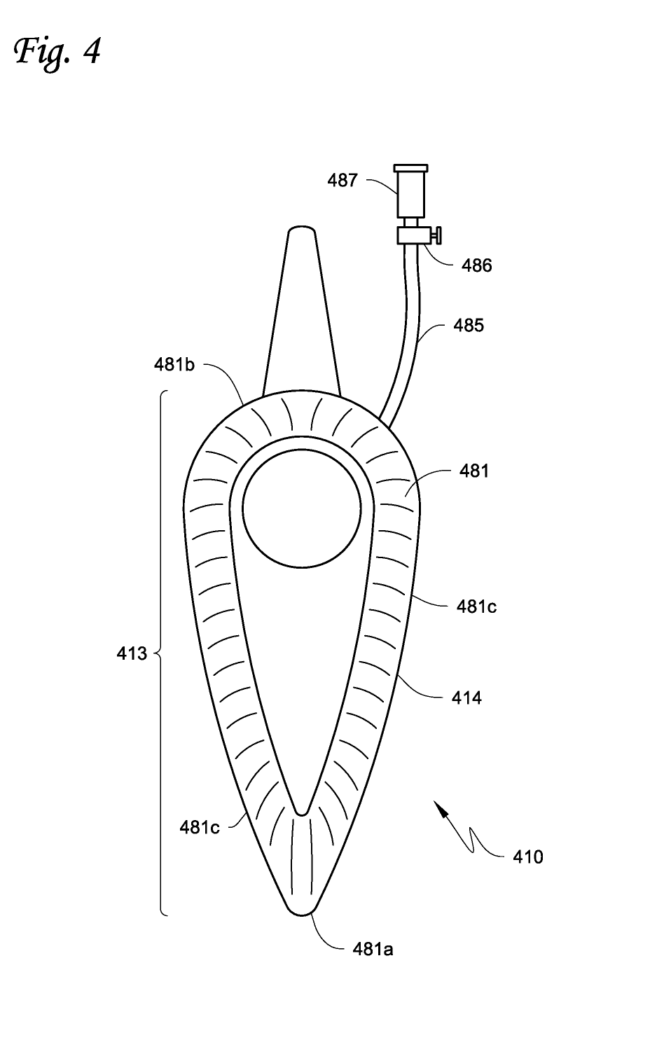

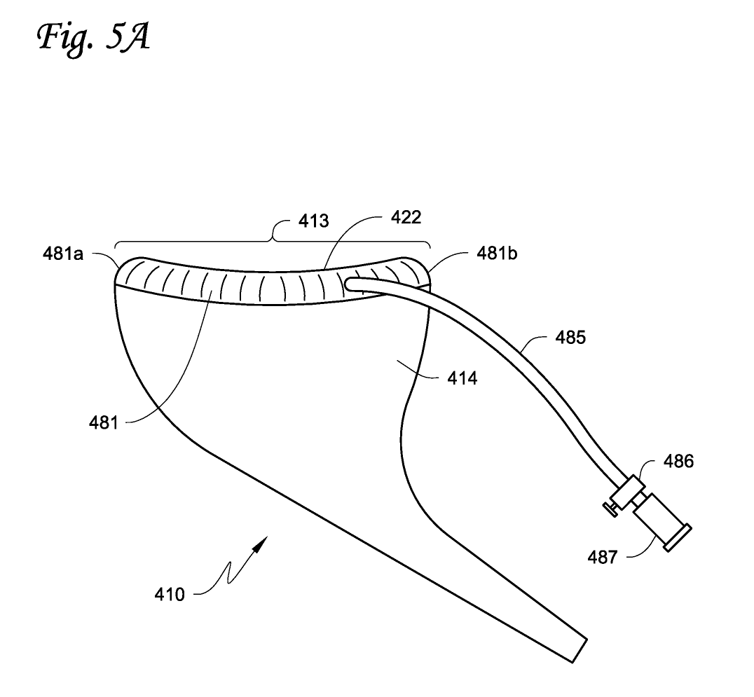

As shown in FIGS. 4, 5A and 5B, in other embodiment of an external voiding apparatus 410, an air bladder 481 is disposed on a container 414. The air bladder 481 generally follows the peripheral of the opening 413 of the container 414. The air bladder 481 includes a superior end 481b configured to align generally with the mons veneris of the female anatomy and an inferior end 481a configured to align generally with the perineal region of the female anatomy. Extending between the superior end 481b and the inferior end 481a of the air bladder are two sides 481c that are arranged and configured to generally align along the labium majus of the female anatomy. The entire air bladder 481 may have just one internal air chamber, or may include a plurality of air chambers at different sections of the air bladder 481. The internal air chamber is connected to a tube 485 that has a valve 486. Air inside the internal air chamber may be released through the tube 485 and the valve 486. Air may also be added into the internal air chamber via a pump 487 through the valve 486. By increasing or reducing air inside the internal air chamber, the air pressure inside the air chamber and therefore the hardness of the air bladder 481 may be regulated. The valve 486 may be air-tight when closed so that the air pressure inside the internal air chamber may be maintained. The air bladder 481 allows a user to regulate the hardness of the air bladder 481 to fit her needs and helps comfortably wear the external voiding apparatus 410 for long period of time. The air bladder 481 may be made from nitrile rubber or thermal plastic.

The shape of the air bladder 481 may be configured to generally follow the anatomy of female vulval region, including the tissues of the mons veneris, labium majus and the tissue of the perineum. In some embodiments, the air bladder 481 is configured to be able to form a generally air-tight seal with the vulval region. In some embodiments, an adhesive, such as a pressure sensitive adhesive, may be applied on a surface 422 of the air bladder 481 that contacts the female vulval region directly to facilitate the formation of the air-tight seal with the vulval region in operation. In practice, a user may place the air bladder 481 against her vulval region directly.

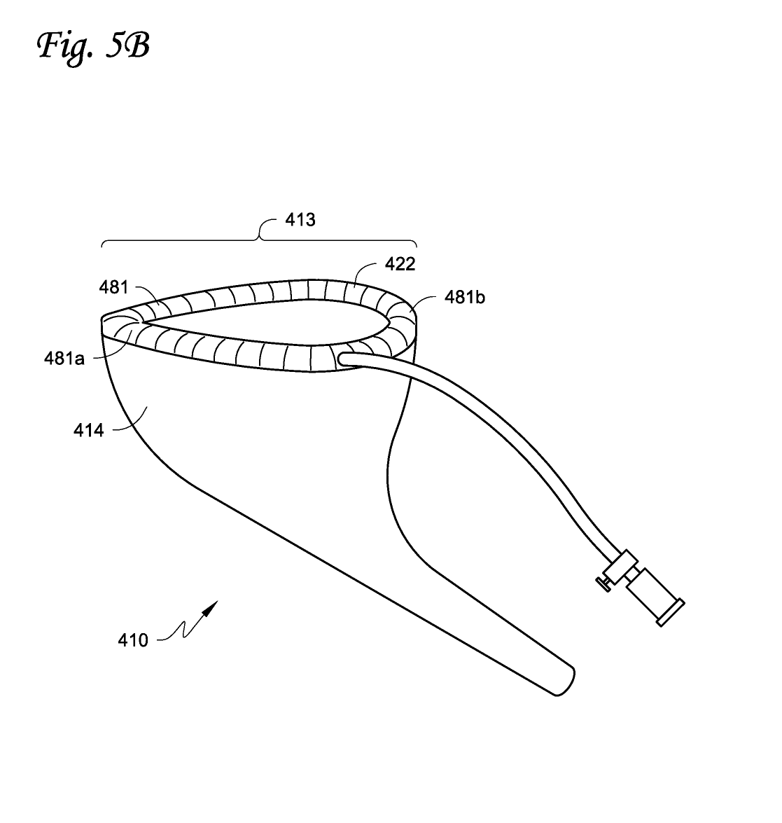

As shown in FIG. 6, in another embodiment of an external voiding apparatus 610, a surface 620 that is configured to generally follow the anatomy of a female's vulval region may have a raised ridge 628, substantially resembling a rib-like structure or protrusion that is preferably disposed on the surface 620 and about the perimeter of the outer surface of the container 614. The raised ridge 628 is adjacent an adhesive 622. The surface 620 may extend inwardly from the outer surface and perimeter of the container 614. In operation, the raised ridge 628 and the inwardly-extended surface 620 may prevent urine from splashing out from the open side 613 of the urine voiding apparatus 600. An air bladder 681 may be disposed underneath, preferably immediately underneath, the inwardly extended surface 620, and supports the surface 620. The air bladder 681 may have a valve 689, from which air may be added or released from the air bladder 681. The valve 689 may also be air-tight so that the air pressure inside the air bladder 681 may be maintained. In operation, a user may regulate the hardness of the air bladder 681 by increasing or decreasing air inside the air chamber (not shown) of the air bladder 681, and therefore regulate the firmness of the surface 620 when the surface 620 is positioned against a user's vulval region. The internally positioned air chamber 681 may also help maintain the shape of the container 614 in operation when the vacuum effect is applied.

The size of the reservoir 650 may vary. Because of the vacuum effect created in the internal space of the external voiding apparatus 610, urine released by the user may be drawn into a vacuum device, such as the vacuum device 280 in FIG. 2.

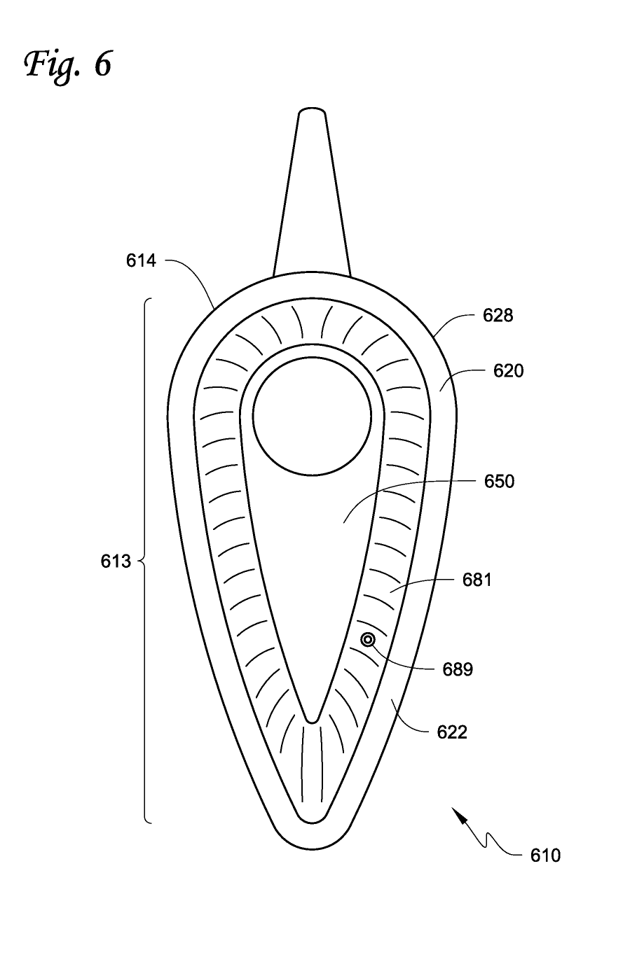

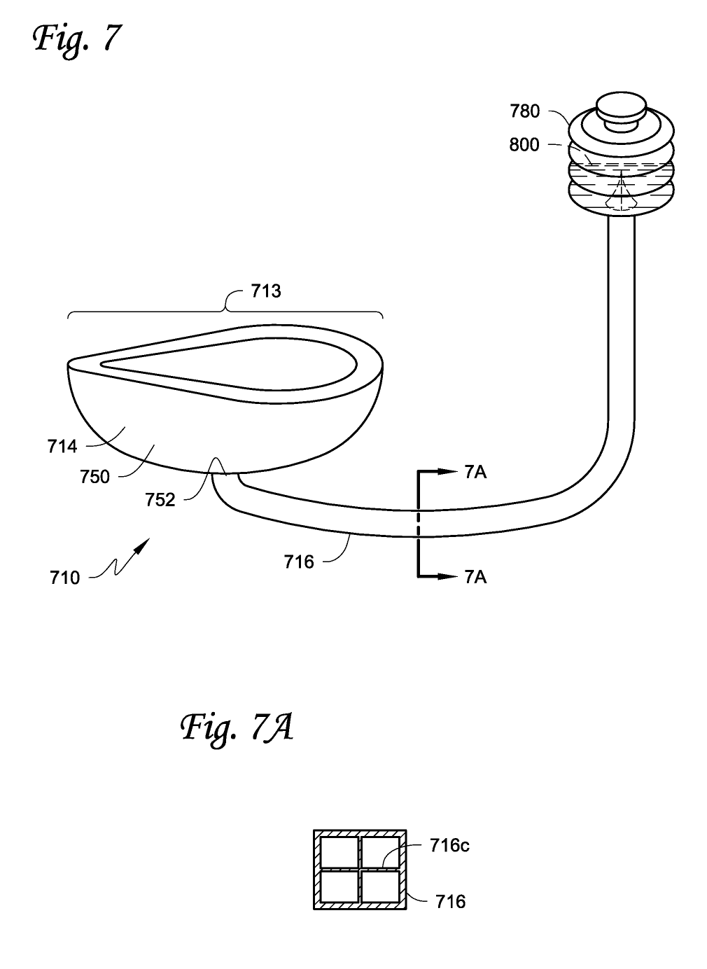

The container may have a low profile or reduced size, such as another embodiment of an external voiding apparatus 710 as shown in FIG. 7. The external voiding apparatus 710 include a reservoir 750, a conduit 716 that is in fluid communication with a vacuum device 780. The size of the reservoir 750 may be relatively small compared to a conventional external voiding apparatus.

In a conventional external voiding apparatus, the drainage of the urine collected in a container may be driven by gravity. Therefore, the urine collected in the container may take some time to be drained. In the embodiments as disclosed herein, the urine is removed by the vacuum effect, therefore removing the urine from the container is relatively fast. Accordingly, the container, such as the container 714 in FIG. 7, can be relatively shallow or small compared to the conventional external voiding apparatus.

The conduit 716 may be positioned very close to the vulval region in use. The conduit 716 may also be oriented to any direction. For example, as illustrated, the orientation of the conduit 716 may be generally parallel to an open side 713. The relatively small size of the container 714 and the flexibility of orienting the conduit 716 make it easier for a user to wear and position the external voiding apparatus 710. It may also help a user wear the external voiding apparatus 710 comfortably for long time.

As shown in FIG. 7, because that vacuum effect is used to draw urine released by a user to the vacuum device 780, the vacuum device 780 does not have to be positioned on the opposite side of the open side 713 relative to an opening 752. In practice, a user may position the vacuum device 780 at the same side as the open side 713 relative to the opening 752. For example, a user may carry the vacuum device 780 on a waist-belt. This may help a user access the vacuum device 780 easily when wearing the apparatus, and help the user wear, remove, drain or exchange vacuum device 780 easily. By eliminating the need to wear the vacuum device 780 around the user's thigh, the user's mobility may also be enhanced.

The materials for the apparatus 710 may vary. Any materials that can be made air impermeable are suitable. For example, the container 714 and conduit 716 may be made from silicon or thermal plastic. It is preferred that the materials and the structures of the container 714 and conduit 716 may be configured to withstand the vacuum effect created by the vacuum device 780 so that both components do not collapse when the vacuum effect is created. If soft materials are used, the structure of the apparatus 710 may incorporate designs to increase the structural rigidity of the components. For example, as shown in FIG. 7A, internal rib-like structure 716c may be used to increase the rigidity of the conduit 716. In addition, the outline of the conduit 716 may also incorporate a shape that helps withstand the vacuum effect. For example, the outline of the conduit 716 may be a rectangular shape as shown in FIG. 7A.

It is to be appreciated that the embodiments as disclosed in FIGS. 4, 5A, 5B, 6 and 7 can be connected to a vacuum device (e.g. the vacuum device 280 as shown in FIG. 2). The embodiments as disclosed in FIGS. 4, 5A, 5B, 6 and 7 can also be connected to other urine collection devices or apparatuses.

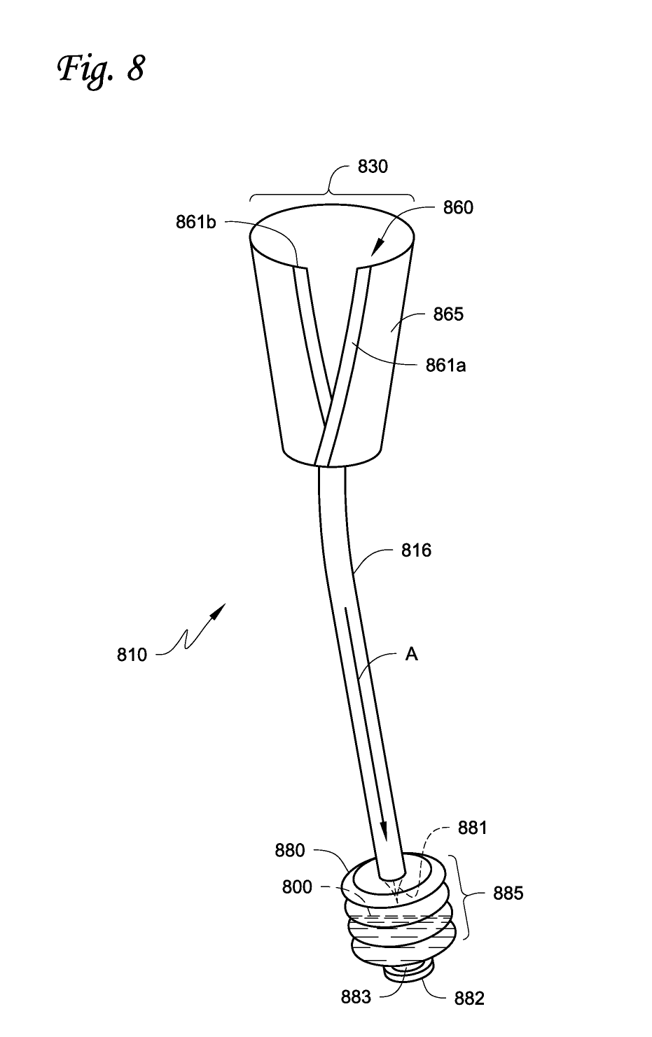

An external voiding apparatus may also be configured for male use. As shown in FIG. 8, one embodiment of a male external voiding apparatus 810 is disclosed. An open side 830 of the external voiding apparatus 810 includes a sheath 865 that is generally configured to surround a male's penis. The material of the sheath 865 may be made air impermeable. The sheath 865 may have an internal reservoir 860. The internal reservoir 860 may be connected to a conduit 816, and the conduit 816 may be connected to a vacuum device 880, so that the reservoir 860, the conduit 816 and the vacuum device 880 form a generally air-tight urine passage. The sheath 865 may also have flaps 861a and 861b. The flaps 861a and 861b are configured to be able to form a resealable air-tight closure between them. For example, a pressure sensitive gel may be applied to the flaps 861a and/or 861b. When the flaps 861a and 861b are pressed against each other, the pressure sensitive gel may form an air-tight closure between the flaps. Hook-and-loop type closures may also be used.

In operation, a male user may separate the flaps 861a and 861b so that he can put the sheath 865 easily around his penis externally. After placing the sheath 865 around his penis, the user may press the flaps 861a and 861b against each other to form an air-tight closure. The closed sheath 865 may form a generally air-tight seal between the sheath 865 and the penis. The user may then active the vacuum device 880 to create a vacuum effect inside the reservoir 860, conduit 16 and the vacuum device 880. Urine 800 released by the penis may be drawn toward the vacuum device 880 through a drainage passage A that is formed by the reservoir 860, conduit 816 and the vacuum device 880, and collected and stored in the vacuum device 880.

The vacuum device 880 may have a similar structure as the vacuum device described above for female apparatuses. Generally, the vacuum device 880 may be activated by compressing an elastic portion 885 of the vacuum device 880 to decrease the volume of the vacuum device 880, as urine 800 is collected in the vacuum device 880, the volume of the vacuum device 880 increases and the vacuum device 880 returns to a non-activated state. Urine 800 collected in the vacuum device 880 may be drained through a drainage port 883 by removing the cap 882 of the drainage port 883. The vacuum device 880 may also have a one-day valve 881 to prevent urine 800 collected in the vacuum device 880 from flowing back to the conduit 816.

The material for the sheath 865 may be elastic, so that the air-tight seal between the sheath 865 and penis may be maintained even when the size of penis changes in use of the external voiding apparatus 810. Elastic materials may also facilitate the placement of the sheath 865 around the penis in use.

With regard to the foregoing description, it is to be understood that changes may be made in detail, especially in matters of the construction materials employed and the shape, size and arrangement of the parts without departing from the scope of the present invention. It is intended that the specification and depicted embodiment to be considered exemplary only, with a true scope and spirit of the invention being indicated by the broad meaning of the claims.

1. A external voiding apparatus comprising:
a container configured to receive urine;
a top portion disposed on the container, the top portion configured to fit a perineal region of a human being;
a conduit being in fluid communication with the container;
a vacuum device being in-line with the conduit, wherein the vacuum device is configured to receive urine; and
the vacuum device has an elastic portion, deforming the elastic portion provides a vacuum effect in the conduit.

2. (canceled)

3. The external voiding apparatus of claim 1, wherein the top portion includes an air bladder.

4. The external voiding apparatus of claim 3, wherein the air bladder further comprises an air chamber.

5. The external void apparatus of claim 2, wherein the elastic portion of the vacuum device includes a plurality of bellows.

6. The external voiding apparatus of claim 4, wherein the air chamber has a valve that is configured to allow air to be pumped into or released from the air chamber.

7. The external voiding apparatus of claim 4, wherein pumping air into the air chamber increases a hardness of the air bladder; releasing air out of the air chamber decreases a hardness of the air bladder.

8. The external voiding apparatus of claim 1, wherein the top portion is configured to include an adhesive that contacts the perineal region of a human being.

9. The external voiding apparatus of claim 1, wherein the top portion is configured to fit a female vulva region.

10. The external voiding apparatus of claim 1, wherein the top portion is configured to fit a male penis.

11. The external voiding apparatus of claim 1, wherein the top portion includes a sheath that is configured to fit a male penis.

12. The external voiding apparatus of claim 11, wherein the sheath further comprises first and second closure flaps that are configured to stick to each other.

13. The external voiding apparatus of claim 12, wherein the first and second closure flaps further comprises hook-and-loop fasteners.

14. The male external voiding apparatus as claim 12, wherein the first closure flap comprises an adhesive.

15. A method to voiding urine, comprising:
providing sealing between a perineal region of a human being and an open side of a container that is configured to receive urine;
providing a vacuum effect in a drainage passage by a vacuum source positioned in line with the drainage passage, wherein the drainage passage is in fluid communication with the container; and
directing the urine collected in the container toward the vacuum source through the drainage passage.
